(19) United States
(12) Patent Application Publication
(10) Pub. No.: US 2009/0130206 A1
(43) Pub. Date: May 21, 2009

(54) Controlled Release Compositions of an Antidepressant Agent

(76) Inventors: Umesh Nandkumar Khatavkar, Hyderabad (IN); Sudarshan Nimbalkar, Hyderabad (IN); Hidaytulla Shamshuddin Aga, Hyderabad (IN); Kishor Dattatray Deo, Hyderabad (IN); Sivakumaram Meenakshisunderam, Hyderabad (IN)

Correspondence Address: Jay Akhave, 2058 N Mills Ave, #612, Claremont, CA, 91711, US

(21) Appl. No.: 12/227,149

(22) PCT Filed: May 7, 2007
(86) PCT No.: PCT/IB2007/001291

§ 371 Date: Nov. 8, 2008

(30) Foreign Application Priority Data
May 9, 2006 (IN) 819/CHE/2006

(51) Int. Cl. A61K 9/24; A61K 31/445; B05D 3/00

(52) U.S. Cl. 424/471; 514/320; 427/2.21

(57) ABSTRACT

The present invention relates to controlled release compositions comprising an anti-depressant compound. More particularly, the present invention relates to controlled release compositions comprising paroxetine hydrochloride.

### FIELD OF THE INVENTION

The present invention relates to controlled release compositions comprising an anti-depressant compound. More particularly, the present invention relates to controlled release compositions comprising paroxetine hydrochloride.

### BACKGROUND OF THE INVENTION

Paroxetine as disclosed in U.S. Pat. No. 4,007,196, is a serotonin re-uptake inhibitor and chemically, paroxetine is (−)-trans-3-[(1,3-benzodioxol-5-yloxy)methyl]-4-(4-fluorophenyl)piperidine. It is useful for the treatment of psychiatric problems such as depression, parkinson's disease, anxiety disorders, obsessive-compulsive disorders, panic disorder and post-traumatic stress disorder. Paroxetine is commercially marketed in the form of hydrochloride salt as immediate release and controlled release tablets by GlaxoSmithKline under the trade name Paxil® and Paxil CR in the US and Seroxat® in other countries.

Administration of conventional dosage form requires frequent dosing to maintain reasonably stable plasma concentrations. However, frequent dosing results in increased frequency of side effects such as nausea and vomiting and leads to poor patient compliance. In view of these problems controlled-release pharmaceutical composition comprising paroxetine has been developed which results in reduced frequency of dosing, improved patient compliance and reduced frequency of side effects.

Given below are the patents/patent publications, which disclose controlled release compositions of paroxetine.

U.S. Pat. No. 4,839,177 and U.S. Pat. No. 5,422,123 disclose controlled release dosage forms consisting of a deposit core of defined geometrical form containing the active substance, polymer substances which swell on contact with aqueous liquids and polymer substances with gelling properties, and a support platform coat partially coating the deposit core wherein the support platform consisting of polymer substances which are slowly soluble and/or slowly gellable in aqueous liquids, plasticizing substances, and other adjuvants.

U.S. Pat. No. 6,548,084 discloses the bilayer enteric-coated tablet formulation of Paroxetine hydrochloride where the active layer is having a defined geometric form along with support platform. It is further disclosed that the said controlled and delayed release formulation containing paroxetine give rise to reduction in the side effects associated with the swallow tablets. The dosage forms are formulated in a manner such that release of active substance is affected predominantly in the small intestine.

US 2005/0266082 discloses a hydrophobic matrix comprised of paroxetine hydrochloride and a lipid component is provided, wherein the matrix also preferably contains hydrophilic polymers and a method of making such a composition by melt granulating paroxetine HCl with a molten binder comprising a lipid component.

US 2006/0039975 discloses a controlled release dosage forms comprising release-retarding materials other than hydroxypropyl methylcellulose and also methods of wet granulating controlled release paroxetine dosage forms.

WO 2005/034954 discloses a modified release composition comprising paroxetine, microcrystalline cellulose, at least one modified release polymer, and one more pharmaceutically acceptable inert excipients, where in the pharmaceutical composition is prepared by a wet granulation technique.

WO 2005/107716 discloses a controlled release tablet comprising a core consisting of paroxetine, at least one rate controlling hydrophilic polymer, a diluent, a binder and a lubricant; and (ii) a coating consisting of an enteric polymer and a plasticizers.

WO 2006/123364 discloses an oral drug delivery system comprising a core comprising active ingredient, and a coating surrounding the core, said coating includes a water-insoluble cellulose derivative, preferably ethyl cellulose and a pH-dependent polymer, preferably a methacrylic acid derivative.

WO 2007/011139 discloses sustained-release tablet comprising paroxetine hydrochloride, a highly viscous polymer, a low viscous hydroxy propylmethylcellulose and a pharmaceutically acceptable excipient.

WO 2007/015270 discloses a controlled release composition comprising a) a core comprising the active ingredient, one or more controlled release polymer(s) and one or more pharmaceutically acceptable excipients; and optionally b) a coating comprising one or more controlled release polymers.

WO 2007/029087 discloses a controlled release multiple unit dosage form comprising: (i) an inert core unit comprising ethyl cellulose and optionally one or more water-soluble or water-swellable excipients; (ii) an active layer on the surface of the inert core comprising one or more active ingredients and one or more hydrophilic polymers; (iii) and polymeric layer over the active layer, wherein the polymeric layer is effective for controlling or modifying the release of active ingredient.

WO 2007/035816 discloses a composition comprising: a) a compressed core containing a mixture comprising: paroxetine or a salt thereof, ethylcellulose, and a hydroxypropyl methylcellulose polymer having a nominal viscosity about 5 to about 100 cP; or paroxetine or a salt thereof and a combination of a hydroxypropyl methylcellulose polymer having a nominal viscosity about 25,000 to about 100,000 cP and a hydroxypropyl methylcellulose polymer having a nominal viscosity about 5 to about 100 cP; and b) a coating over the core comprising an acid-resistant polymer.

In order to reduce the side effects associated with administration of swallow tablets, the above patents/patent publications disclose various controlled release dosage forms of paroxetine. However, still there is a need to develop controlled release dosage form, which would be bioequivalent to the marketed controlled release dosage form. The inventors of the present invention during their efforts to develop controlled release dosage form found that by making a dosage form which release 15% of paroxetine in first 2 hours and releasing the remaining amount of paroxetine in a controlled manner can effectively minimizes the incidence of side effects by avoiding the release of paroxetine in the stomach.

### OBJECTIVE OF THE INVENTION

Accordingly, the main objective of present invention is to provide controlled release dosage forms of paroxetine.

Another objective of the present invention is to provide controlled release dosage forms of paroxetine in such a way that it will comply with the reference product in terms of in vivo parameters like Cₘₐₓ, tₘₐₓ and AUC and in vitro parametes like dissolution etc.

Yet another objective of the present invention is to provide process for the preparation of controlled release dosage forms of paroxetine.

### SUMMARY OF THE INVENTION

The present invention provides controlled release dosage form comprising:

(a) controlled release matrix core comprising about 85% of paroxetine,
(b) first coating layer comprising enteric coating polymer over the controlled release core and
(c) second coating layer comprising about 15% of paroxetine over the enteric-coated core.

### DETAILED DESCRIPTION OF THE INVENTION

In yet another embodiment of the present invention, the controlled release matrix core comprising paroxetine further comprise one or more pharmaceutically acceptable excipients such as diluents, binders, release retarding polymers, glidants, lubricants and the like.

In yet another embodiment, the core of the present invention may be in the form of spheroids/tablet/mini tablets and the like.

In yet another embodiment of the present invention, second coating comprising 15% of paroxetine is released within 2 hours after the administration and the core comprising 85% of paroxetine is released in a controlled manner at a pH 5.5 and above.

Suitable diluents used according to the present invention are selected from calcium phosphate-dibasic, calcium carbonate, lactose, sucrose, cellulose-microcrystalline, cellulose powdered, calcium silicate, starch, starch pregelatinized, polyols such as mannitol, sorbitol, lactitol, xylitol, maltitol, sucrose and combinations thereof.

Suitable binders used according to the present invention are selected from hydroxy propyl cellulose, hydroxypropyl methylcellulose, gelatin, hydroxy ethyl cellulose, povidone, polyvinyl alcohol, copovidone, ethylcellulose, starch and methylcellulose or a combination thereof.

Suitable release retarding polymers used according to the present invention are selected from hydroxypropyl methylcellulose, hydroxypropyl cellulose, hydroxyethyl cellulose, carbopol, alginic acid salts, xanthan gum, ethyl cellulose, cross-linked carboxymethyl cellulose or its salts, polyvinyl alcohol, pH independent polymethacrylates, polyvinyl acetate and combinations thereof.

Suitable glidants used according to the present invention are selected from magnesium trisilicate, talc, tribasic calcium phosphate, glyceryl monostearate, glyceryl stearate and colloidal silica or a combination thereof.

Suitable lubricants used according to the present invention are selected from calcium stearate, magnesium stearate, hydrogenated vegetable oil, stearic acid, sodium stearyl fumarate or a combination thereof.

The term paroxetine according to the present invention includes its pharmaceutically acceptable salts such as hydrochloride, hydrobromide and the like.

The presence of enteric coating prevents release of paroxetine until the dosage form reaches the small intestine, thereby reduces the side effects associated with the immediate release tablets.

Suitable enteric coating polymers used according to the present invention are selected from the group consisting of cellulose acetate phthalate, polyvinyl acetate phthalate, methacrylic acid copolymer, cellulose acetate trimellitate, shellac, hydroxypropylmethyl cellulose phthalate, hydroxypropylmethylcellulose acetate succinate or combinations thereof. In a preferred embodiment, the enteric coating has a thickness from about 6% to about 16% by weight of the final dosage form.

In yet another embodiment of the present invention, the first coating layer comprising enteric coating polymer further comprise one or more excipients such as plasticizers, anti-tacking agent and the like.

In yet another embodiment of the present invention, the second coating layer comprising 15% of paroxetine further comprises excipients such as diluent, binder, plasticizer and anti tacking agent.

The coating according to the present invention is applied by dissolving/dispersing the excipients in solvents such as isopropyl alcohol, water, acetone, ethanol, methylene dichloride and the like or mixtures thereof.

Suitable plasticizers used according to the present invention are selected from diethyl phthalate, di butyl phthalate, cetyl alcohol, polyethylene glycol-4000, triethyl citrate, triacetin, propylene glycol and the like.

Suitable anti-tacking agents used according to the present invention are selected from talc, magnesium stearate and the like or a mixture thereof.

In yet another embodiment of the present invention, the controlled release dosage form of the present invention is optionally coated with third coating comprising polymers selected from the group consisting of ethyl cellulose, hydroxypropyl cellulose and hydroxypropylmethylcellulose and the like or mixtures thereof; diluents and plasticizers.

Ethyl cellulose is a hydrophobic polymer and is commercially available under different trade names such as Aquacoat, Aquacoat ECD, Aqualon, Ethocel and Surelease.

The tablet dosage form of the present invention may optionally be coated with film coating materials.

In yet another embodiment, there is provided a process for the preparation of controlled release dosage form comprising

(a) controlled release matrix core comprising about 85% of paroxetine,
(b) first coating layer comprising enteric coating polymer over the core and
(c) second coating layer comprising about 15% of paroxetine over the enteric-coated core which comprises the steps of
i) preparing a controlled release matrix core comprising 85% of paroxetine and one or more pharmaceutically acceptable excipients,
ii) coating the core with enteric coating composition comprising enteric polymer,
iii) preparing a coating composition comprising 15% of paroxetine and one or more hydrophilic & or hydrophobic polymers,
iv) applying the coating composition prepared in step (iii) to the enteric-coated core and
v) optionally coating the coated core obtained in step (iii) with third coating composition.

The different formulation processes that can be employed for making the core is dry granulation (slugging, compaction), wet granulation, and direct dry blend filling, when the core is a tablet.

The following examples further exemplify the invention and are not intended to limit the scope of the invention. It is obvious to those skilled in the art to find out the composition for other dosage forms and substitute the equivalent excipients as described in this specification or with the one known to the industry.

### EXAMPLE 1

### Controlled Release Tablet Dosage Form of Paroxetine HCl 37.5 ml

| S. No. | Ingredients | Mg/Tab |
|---|---|---|
| 1 | Paroxetine hydrochloride hemihydrate | 36.269 |
| 2 | Lactose monohydrate | 101.004 |
| 3 | Dicalcium phosphate dihydrate | 12.227 |
| 4 | Colloidal silicon dioxide | 1.000 |
| 5 | Hydroxypropylmethylcellulose | 20.000 |
| 6 | Polyvinyl pyrrolidone | 3.000 |
| 7 | Purified water | QS |
| 8 | Magnesium stearate | 1.500 |
| | Coating I | |
| 1 | Eudragit L 30 D 55 | 11.540 |
| 2 | Triethyl citrate | 1.160 |
| 3 | Talc | 2.300 |
| 4 | Purified Water | QS |
| | Coating II | |
| 1 | Paroxetine hydrochloride hemihydrate | 6.399 |
| 2 | Hydroxypropylmethylcellulose | 3.601 |
| 3 | Triethylcitrate | |
| 4 | Purified water | QS |
| 5 | Isopropyl alcohol | QS |
| | Coating III | |
| | Opadry Blue | 4.000 |
| | Purified Water | QS |
| | Final Coated Tablet Weight | 204.000 |

### EXAMPLE 2 AND 3

| | | Mg/Tab | |
|---|---|---|---|
| S. No. | Ingredients | Example 2 | Example 3 |
| 1 | Paroxetinehydrochloride hemihydrate | 24.178 | 24.178 |
| 2 | Lactose monohydrate | 104.799 | 67.671 |
| 3 | Dicalcium phosphate dihydrate | 11.022 | 8.151 |
| 4 | Colloidal silicon dioxide | 0.902 | 0.667 |
| 5 | Hydroxypropylmethylcellulose | 18.032 | 13.333 |
| 6 | Polyvinyl pyrrolidone | 2.705 | 2.000 |
| 7 | Purified Water | QS | QS |
| 8 | Magnesium stearate | 1.362 | 1.000 |
| | Coating I | | |
| 1 | Eudragit L 30 D 55 | 10.528 | 7.693 |
| 2 | Triethyl citrate | 1.094 | 0.769 |
| 3 | Talc | 2.079 | 1.538 |
| 4 | Purified water | QS | QS |
| | Coating II | | |
| 1 | Paroxetinehydrochloride hemihydrate | 4.267 | 4.267 |
| 2 | Hydroxypropylmethylcellulose | 3.938 | 1.733 |
| 3 | Triethylcitrate | | |
| 4 | Purified water | QS | QS |
| 5 | Isopropyl alcohol | QS | QS |
| | Coating III | | |
| | Opadry Blue | 3.000 | 3.000 |
| | Purified Water | QS | QS |
| | Final Coated Tablet Weight | 184.000 | 136.000 |

The processing steps involved were as given below:
i) sifted and blended paroxetine hydrochloride hemihydrate, lactose monohydrate, dicalcium phosphate dihydrate, hydroxypropyl methylcellulose in a granulator,
ii) dissolved polyvinylpyrrolidone in purified water and granulated the contents of step 1 in rapid mixer granulator,
iii) dried the wet mass and lubricated with magnesium stearate,
iv) compressed the lubricated blend to obtain controlled release tablet core,
v) a dispersion of Eudragit, triethylcitrate and talc in purified water was prepared and coated on to the controlled release tablet core,
vi) a solution of paroxetine hydrochloride hemihydrate, hydroxypropyl methylcellulose and triethylcitrate in a mixture of isopropyl alcohol and water was prepared and coated on to the enteric coated tablet core and,
vii) tablets of step (vi) were then coated with a solution/suspension of opadry in water.

### EXAMPLE 4 AND 5

| | | Mg/Tab | |
|---|---|---|---|
| S. No. | Ingredients | Example 4 | Example 5 |
| 1 | Paroxetinehydrochloride hemihydrate | 36.267 | 24.179 |
| 2 | Lactose monohydrate | 101.004 | 66.659 |
| 3 | Dicalcium phosphate dihydrate | 12.227 | 8.81 |
| 4 | Colloidal silicon dioxide | 1.000 | 0.664 |
| 5 | Hydroxypropylmethylcellulose | 20.000 | 13.334 |
| 6 | Polyvinyl pyrrolidone | 3.000 | 1.994 |
| 7 | Purified water | QS | QS |
| 8 | Magnesium stearate | 1.500 | 1.003 |
| | Coating I | | |
| 1 | Eudragit L 30 D 55 | 12.115 | 8.07 |
| 2 | Triethyl citrate | 1.211 | 0.81 |
| 3 | Talc | 2.423 | 1.61 |
| 4 | Purified Water | QS | QS |
| | Coating II | | |
| 1 | Paroxetinehydrochloride hemihydrate | 6.401 | 4.266 |
| 2 | Hydroxypropylmethylcellulose | 5.599 | 2.40 |
| 3 | Triethylcitrate | | |
| 4 | Talc | 2.001 | 1.334 |
| 5 | Purified water | QS | QS |
| 6 | Isopropyl alcohol | QS | QS |
| | Coating III | | |
| 1 | Aquacoat ECD | 3.763 | 2.51 |
| 2 | Hydroxypropylmethylcellulose | 1.003 | 0.67 |
| 3 | Triethylcitrate | 1.254 | 0.836 |
| 4 | Purified water | QS | QS |
| | Coating IV | | |
| | Opadry Blue | 4.180 | 2.784 |
| | Purified Water | QS | QS |
| | Final Coated Tablet Weight | 213.01 | 142.003 |

The processing steps involved were as given below:
i) sifted and blended paroxetine hydrochloride hemihydrate, lactose monohydrate, dicalcium phosphate dihydrate, hydroxypropyl methylcellulose in a granulator,
ii) dissolved polyvinylpyrrolidone in purified water and granulated the contents of step 1 in rapid mixer granulator,
iii) dried the wet mass and lubricated with magnesium stearate,
iv) compressed the lubricated blend to obtain controlled release tablet core,
v) a dispersion of Eudragit, triethylcitrate and talc in purified water was prepared and coated on to the controlled release tablet core,
vi) a solution of paroxetine hydrochloride hemihydrate, hydroxypropyl methylcellulose and triethylcitrate in a mixture of isopropyl alcohol and water was prepared and coated on to the enteric coated tablet core,
vii) a dispersion of Aquacoat ECD 30, triethylcitrate & hydroxypropyl methylcellulose in water was prepared and coated on to drug layered core and
viii) tablets of step (vii) were then coated with a solution/suspension of opadry in water.
Table 1 given below shows the comparative dissolution profile of Paroxetine hydrochloride controlled release tablets of the present invention (test) & Paxil CR® tablets (Reference) carried out in 750 ml 0.1 N HCl for 2 hours followed by 0.05 M, 1000 mL, Tris Buffer pH 7.5 as medium for further 7 hours using Apparatus USP II (Paddle), @ 150 rpm speed. The release profile (% of drug released) is given in table 1.

TABLE 1

| | PAXILCR 37.5 | Example No. | | | | |
|---|---|---|---|---|---|---|
| Time | (B. No 3105P08) | 1 | 2 | 3 | 4 | 5 |

| | |
|---|---|
| (hours) | % release of paroxetine |

| | | | | | | |
|---|---|---|---|---|---|---|
| 0 | 0 | 0 | 0 | 0 | 0 | 0 |
| 2 | 0 | 11 | 15 | 15 | 15 | 15 |
| 3 | 4 | 15 | 17 | 20 | 18 | 17 |
| 4 | 18 | 25 | 23 | 28 | 26 | 23 |
| 5 | 40 | 43 | 39 | 40 | 40 | 43 |
| 6 | 60 | 63 | 61 | 59 | 58 | 61 |
| 7 | 77 | 78 | 78 | 76 | 76 | 77 |
| 8 | 93 | 90 | 88 | 80 | 89 | 88 |
| 9 | 95 | 92 | 92 | 95 | 97 | 93 |

We claim:

1. A controlled release paroxetine dosage form comprising:
(a) a controlled release matrix core comprising about 85% of the total paroxetine dosages
(b) a first coating layer comprising enteric coating polymer over the controlled release matrix core and
(c) a second coating layer comprising about 15% of the total paroxetine dosage over the enteric-coated core.

2. The dosage form of claim 1, wherein the controlled release matrix core is in the form of a spheroid, tablet or a mini tablets.

3. The controlled release matrix core as claimed in claim 1, further comprises one or more pharmaceutically acceptable excipients such as diluent, binder, release retarding polymer, glidant and lubricant.

4. The controlled release matrix core as claimed in claim 3, wherein the diluent is selected from calcium phosphate-dibasic, calcium carbonate, lactose, sucrose, cellulose-microcrystalline, cellulose powdered, calcium silicate, starch, starch pregelatinized, polyols such as mannitol, sorbitol, lactitol, xylitol, maltitol, sucrose and combination thereof.

5. The controlled release matrix core as claimed in claim 3, wherein the binder is selected from hydroxypropyl cellulose, hydroxypropyl methylcellulose, gelatin, hydroxyethyl cellulose, povidone, polyvinyl alcohol, copovidone, ethylcellulose, starch and methylcellulose and combination thereof.

6. The controlled release matrix core as claimed in claim 3, wherein the lubricant is selected from calcium stearate, magnesium stearate, hydrogenated vegetable oil, stearic acid, sodium stearyl fumarate and combination thereof.

7. The controlled release matrix core as claimed in claim 3, wherein the release retarding polymer is selected from the group consisting of hydroxypropyl methylcellulose, hydroxypropylcellulose, hydroxy ethylcellulose, carbopol, alginic acid salts, xanthan gum, ethylcellulose, cross-linked carboxymethylcellulose or its salts, polyvinyl alcohol, pH independent polymethacrylates, polyvinyl acetate and combination thereof.

8. The dosage form as claimed in claim 1, wherein the enteric coating polymer is selected from the group consisting of cellulose acetate phthalate, polyvinyl acetate phthalate, methacrylicacid copolymer, cellulose acetate trimellitate, shellac, hydroxypropyl methylcellulose phthalate, hydroxypropyl methylcellulose acetate succinate and combination thereof.

9. The dosage form as claimed in claim 1, the first coating layer comprising enteric coating polymer further comprises plasticizer and anti-tacking agent.

10. The dosage form as claimed in claim 9, wherein the plasticizer is selected from diethyl phthalate, dibutyl phthalate, cetyl alcohol, polyethylene glycol-4000, triethyl citrate, triacetin or propylene glycol.

11. The dosage form as claimed in claim 9, wherein the anti-tacking agent is selected from talc or magnesium stearate.

12. The dosage form as claimed in claim 1, wherein the second coating comprising 15% of total paroxetine further comprises excipients such as diluent, binder, plasticizer and anti-tacking agent.

13. The dosage form as claimed in claim 1, further comprise third coating comprising polymers selected from the group consisting of ethyl cellulose, hydroxypropyl cellulose and hydroxypropyl methylcellulose or mixtures thereof, diluent and plasticizer.

14. A process for the preparation of controlled release paroxetine dosage form comprising:
i) preparing a controlled release matrix core comprising 85% of total paroxetine dosage and one or more pharmaceutically acceptable excipients,
ii) coating the core with enteric coating composition comprising enteric polymer,
iii) preparing a coating composition comprising 15% of total paroxetine dosage and one or more hydrophilic and/or hydrophobic polymers,
iv) applying the coating composition prepared in step (iii) to the enteric-coated core and
v) optionally coating the enteric coated core with a third coating composition.
